(19) United States
(12) Patent Application Publication
(10) Pub. No.: US 2023/0358717 A1
(43) Pub. Date: Nov. 9, 2023

(54) CONSUMER ENDPOINT QUALITY CONTROL SYSTEMS

(71) Applicant: Athanasios Demeslis, Almyros (GR)

(72) Inventor: Athanasios Demeslis, Almyros (GR)

(21) Appl. No.: 18/118,442

(22) Filed: Mar. 7, 2023

Related U.S. Application Data
(60) Provisional application No. 63/269,009, filed on Mar. 8, 2022.

(51) Int. Cl. G01N 21/3563; G01N 33/02; B65B 25/00

(52) U.S. Cl. CPC G01N 33/02; B65B 25/001; G01N 21/3563

(57) ABSTRACT

A method for assuring quality control of food products is provided. The method comprises a computer directing a storage device to dispense a first quantity of a food product. The method also comprises the computer directing a quality control device to perform at least one test on the dispensed product. The method also comprises the computer directing a networking device to transmit instructions regarding at least maintaining and monitoring the storage device and quality control device. The method also comprises the computer directing a consumer experience device to provide details describing the at least one test. The method also comprises the computer directing replacement of the storage device upon detecting an empty condition of the storage device, the storage device comprising a flexi-bag. The method also comprises the computer directing the quality control device to measure at least one characteristic of the dispensed food product.

### CROSS REFERENCE TO RELATED APPLICATIONS

The present non-provisional patent application is related to U.S. Provisional Pat. Application No. 63/269,009 filed Mar. 8, 2022, the contents of which are incorporated herein in their entirety.

### FIELD OF THE DISCLOSURE

The present disclosure is in the field of food safety. More particularly the present disclosure provides devices, systems, and methods for real time quality control analysis and reporting at consumer endpoints, such as grocery stores, for determining and providing evidence of quality of food products such as olive oil.

### BACKGROUND

Many consumers are concerned about the quality of their food, and for many types of food, generally have no way of objectively evaluating the quality of the food at the time of purchasing the food. Such consumers may rely on packaging messages and statements provided by the manufacturer or quality control laboratory, which may or may not be accurate at the time of purchase.

Because many foods, for example olive oil products, undergo complex supply chain journeys on their way to the consumer, these foods are at high risk of becoming degraded during storage and transport. As a result, the accuracy of quality control laboratory tests made before entering the supply chain may no longer be reliable and the consumer may unknowingly purchase mislabeled or degraded food products, wasting time and money. This phenomenon may also constitute a health risk for consumers.

Accordingly, there is a need for objective, real-time quality control testing of foods, such as olive oil products, at consumer endpoints for maintaining consumer confidence in the quality of the products.

### SUMMARY

The present disclosure provides improved devices, systems, and methods for real-time quality control analysis of food products and reporting of quality control results at consumer endpoints, such as food retailers. Systems provided herein include a storage feature for storing a food product, a quality control feature for determining and reporting a quality control characteristic of the food product, a networking feature for networked communications as may be needed for maintenance and monitoring of the system, and a consumer experience feature that enables a consumer to effectively interact with the system.

Other objects, features and advantages of the present invention will become apparent from the following detailed description taken in conjunction with the accompanying drawings.

### BRIEF DESCRIPTION OF THE DRAWINGS

Although the characteristic features of the invention will be particularly pointed out in the claims, exemplary implementations of the invention and manners in which they may be made and used may be better understood after a review of the following description, taken in connection with the accompanying drawings, wherein like numeral annotations are provided throughout.

FIG. 1 is a block diagram of a system of food product quality control in accordance with an embodiment of the present disclosure.

FIG. 2 is a schematic drawing of an exemplary real-time food product quality control system in accordance with an embodiment of the present disclosure.

FIG. 3 is a schematic drawing of an exemplary quality control feature in accordance with an embodiment of the present disclosure.

FIG. 4 is a schematic drawing of an exemplary data fusion and data analysis method in accordance with an embodiment of the present disclosure.

### DETAILED DESCRIPTION

Reference is made herein to the attached drawings. Like reference numerals may be used in the drawings to indicate like or similar elements of the description. The figures are intended for representative purposes, are not drawn to scale, and should not be considered limiting.

Unless otherwise defined herein, terms and phrases used in connection with the present disclosure shall have the meanings that are commonly understood by those of ordinary skill in the art. Where a reference is made to a singular noun, whether with or without use of an indefinite or definite article (e.g., “a”, “an”, or “the”), this includes a plural of that noun unless something else is specifically stated.

Furthermore, the terms first, second, third, and the like in the description and in the claims, are used for distinguishing between elements and not necessarily for describing a sequential or chronological order. It is to be understood that the terms so used are interchangeable under appropriate circumstances and that the implementations of the disclosure described herein are capable of operation in other sequences than described or illustrated herein.

As used herein, the term “about” refers to the usual error range for the respective value readily known to the skilled person in this technical field. Reference to “about” a value or parameter herein includes and describes implementations and embodiments that are directed to that value or parameter per se.

A storage compartment (e.g., a 500-liter food-grade flexi-bag) is configured to allow a certain number of transactions (e.g., about 1000 transactions) before a refill of the storage compartment is required. A flexi-bag storage compartment may be considered a consumable that may be replaced with every refill by a support team affiliated with maintenance or support of the system.

The flexi-bag storage compartment may include a tap at a bottom portion thereof, to supply the quality control feature with olive oil samples and to supply consumers with the olive oil food product. The flexi-bag storage compartment may be a recyclable solution that increases the handling capacity of the container due to its reduced weight.

In addition, it may provide advantages compared to a different material container, such as a steel container: cleaning of the container is not required, which greatly reduces complexity and makes automation more feasible; and also, a flexi-bag protects the olive oil from oxygen and oxidation without any additional steps, since vacuum replaces the olive oil instead of it being replaced by atmospheric air, or nitrogen, which would also increase costs and complexity.

In various embodiments, a sensor may be positioned on the output tap to calculate the remaining quantity of olive oil and update a management system, such as an enterprise resource planning (ERP) system executing ERP software. The sensor may also be configured to detect spillage such that the spillage can be corrected by personnel. A cooling system may be configured to contact or embrace the flexi-bag to keep the flexi-bag within a suitable temperature range, which may help to preserve the olive oil’s chemical and physical features to ensure accuracy of QA/QC tests. The cooling system may include refrigerator elements that keep the temperature between about 14 and about 16° C.

For olive oil food products and, potentially, other food products, real-time quality control may be achieved with use of a combination of quality control methods, such as near-infrared (NIR) spectroscopy, electronic nose and electronic tongue, to measure various quality criteria, such as acidity, peroxide value, UV absorption, sensory features (e.g., fruitiness, defects), origin classification and adulteration detection of a sample. For example, in certain instances, if a sample from an olive oil food product that claims to be an “Extra Virgin” olive oil food product is determined to have an acidity that is higher than 0.8%, then it may be reported by the system as being mislabeled or degraded. The system is configured to determine and clarify various grades of olive oil so that the consumer is informed about the quality of the olive oil in real-time.

An Electronic Nose system may comprise an exposure chamber which may include an array of gas sensors with varying selectivity. The exposure chamber’s conditions, such as temperature and humidity will be monitored and controlled through the system’s cooling and cleaning components. A sample of an olive oil food product may traverse the exposure chamber and the sample’s volatile compounds may interact with said sensors. The sensor signals may be received by the computer component for further analysis.

An Electronic tongue system may utilize an array of sensors suitable for analyzing liquids through the concept of global selectivity. This sensory array may be positioned in a relevant exposure chamber. The electronic tongue may consist of an electronic system which may consist of an electrochemical cell, a multiplexor, and a potentiostat for the accurate transmission of the sensor signals to the computer for further analysis.

Commercially available mini-NIR devices are ultra-light, compact, and can be connected to a computer device of the system with reliable connections such as a universal serial bus (USB) connection. From an economic perspective, the use of a mini-NIR device to measure various quality criteria of the olive oil, will increase the one-time cost for initial acquisition of the container. But use of a mini-NIR device may save costs and complexity in the long run, since the core of the QC system can work without any consumables, spare parts, or maintenance for an extended period of time, perhaps more than 20 years.

After applying an incident light through a suitable light source, the mini-NIR will analyze absorption of the olive oil in the wavelength range between about 853 nm and about 2202 nm, which is processed, e.g., by a partial least-squares (PLS) regression analysis, using suitable software installed on the computer device, which may be viewable and/or controllable by local and/or remote maintenance personnel.

After receiving the signals from the relevant quality control systems, the computer, using specialized data fusion and data analysis software, may proceed to integrate the information into relevant and homogenous data sets that may be then analysed for the production of results.

The results may be compared to a known database that may be preinstalled on a storage device of the computer device, that may be created by a plurality of reference measurements obtained through chemometric analysis to determine the values of the quality criteria accurately. The computer device may generally include a processor and the software may be in the form of processor-executable instructions stored on a non-transitory memory (e.g., a non-transitory machine-readable medium).

A small-size cuvette or flowcell may be used to analyze the sample of olive oil after the sample is dispensed from the flexi-bag. The smaller the size of the cuvette, the smaller the quantity of olive oil samples and cleaning agents that will be needed. The minimum possible flowcell size for the mini-NIR device has a volume of 28 microliters.

A sensor system may be necessary to oversee the sampling and cleaning processes. Upon sampling completion, the cuvette and exposure chamber may rotate according to the positioning of the flow pipes and the dry-cleaning system. A cleaning system may be included, which comprises several cleaning system components. To adequately clean a cuvette and exposure chamber after an analysis, distilled water, acetone, and air may be used. For this reason, the system may include a distilled water container, an acetone container, and a waste container, some or all of which may be managed by a support team periodically.

A dry cleaning system may also be needed within the analysis chamber for the purpose of air-drying the cuvette, the exposure chamber, and the flow pipes after cleaning has taken place.

A mechanical automation system and related software may also be needed to facilitate the necessary “if-then” procedures and other computer logic steps, from sampling, analyzing, and cleaning, to supplying customers with olive oil.

A computer coordinates all or part of the processes described above and to connect the container to a support team, for example via Internet connection. The support team may need remote access to the container to oversee its function, schedule maintenance and resupply, and tackle any potential issues on the spot. Such access may enable the support team to receive near real-time data on consumption and revenue generated.

The system may include computer components that configure the system to be connected to the internet, e.g., as an “internet of things” (IOT) device, which is available to potential consumers that are remote from the system. The system may act as a client and be configured to send and receive data over a network connection, e.g., to send and receive data to and from a networked server.

A server may be configured to deliver web and hypertext transfer protocol (HTTP) content representative of operations and results of one or more quality control systems. By visiting a website provided by the server, a consumer would be able to see the location that serves the consumer satisfactorily and would enable the consumer to acquire details about food product quality control systems and results at that location, such as real-time quality results or reports, olive oil origins, specifications, and reviews from other customers.

A display, e.g., a touch-screen display, may be a primary point of interaction between the consumer and the system. The consumer may interact with the system in various ways; the consumer may be able to: determine the quantity they require, view details of a recent analysis, learn more about origins of the olive oil, learn how it was processed, and even see the faces of the people who produced it or learn about their expertise in their field.

In embodiments, the system may be configured to produce all or part of the electricity needed for the system’s processes and functions. This may be beneficial and encourage adoption by food retailers because food retailers would be able to use the system without increasing operational costs due to electricity being pulled from the food retailer’s connection to the power grid.

As many food retail environments are continuously illuminated by strong lighting, the system may include a solar panel and/or a solar power system positioned on the container, such that ambient or artificial light within the retail environment can be harvested and used by the system. A battery may be needed to achieve 100% autonomy of the system, in order to keep the cooling and remote access systems online, and while the food retailer store is closed, e.g., during nighttime, during which time indoor lighting may not be available. The power requirement may not be overly high since many or most food retailers are only closed for approximately 9 hours.

In various implementations, a computer device or a plurality of computer devices may be used as part of the food product quality control system of the present disclosure. Such computer devices may include a bus that connects a plurality of computer components, which may include but may not necessarily limited to a display (e.g., a non-touchscreen display, a touchscreen display, etc.), a processor (e.g., a single core processor, a multi-core processor, etc.), a memory (e.g., a non-transitory machine-readable medium, a non-transitory processor-readable medium, etc.), and an input device, for example a mouse input or a keyboard input.

The processor may be configured to execute processor-executable code, stored on the memory as processor-executable instructions, that configure the processor to perform all or part of an operation or method related to: storing food samples, dispensing food products, monitoring the system for need to refill or failures or leaks or power supply levels, testing of food samples, reporting of test results from the testing of food samples, local or remote system maintenance activities, or consumer engagement activities.

The processor-executable code may be in the form of one or more software modules, whether stored locally and/or remotely to the system, which provide a set of instructions that enable the processor to perform these and/or other functions as a result of the processor executing the instructions in whole or in part, in any suitable order of operation. In addition, while a hardware-software combination system may provide certain advantages for implementation and maintenance of the system, in other instances, the real time food product quality control system may be implemented as a hardware system without any software elements. In this case, the hardware may include suitable logical circuitry elements that are configured for performing all or part of the functionalities or features described for the hardware-software combination systems.

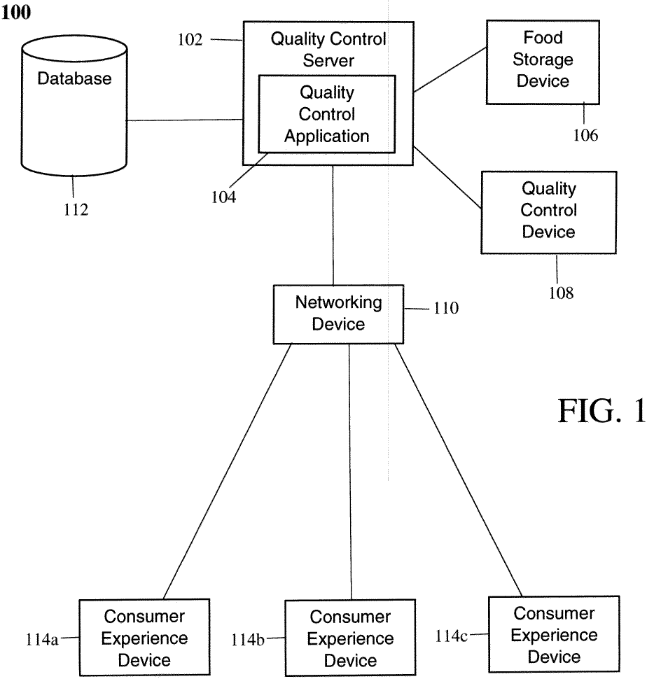

Turning to the figures, FIG. 1 is a block diagram of a system of food product quality control in accordance with an embodiment of the present disclosure. FIG. 1 depicts components and their relationships of a system 100.

The system 100 comprises a quality control server 102 and a quality control application 104, referred to hereinafter respectively for brevity purposes as the server and the application 102. The system 100 also comprises a food storage device 106, a quality control device 108, a networking device 110, and a database 112. The system 100 also comprises consumer experience devices 114a-c. In embodiments, the consumer experience devices 114a-c may be laptop computer, desktop computers, mobile device, or touch-screen display device in the possession of consumers of food as described herein.

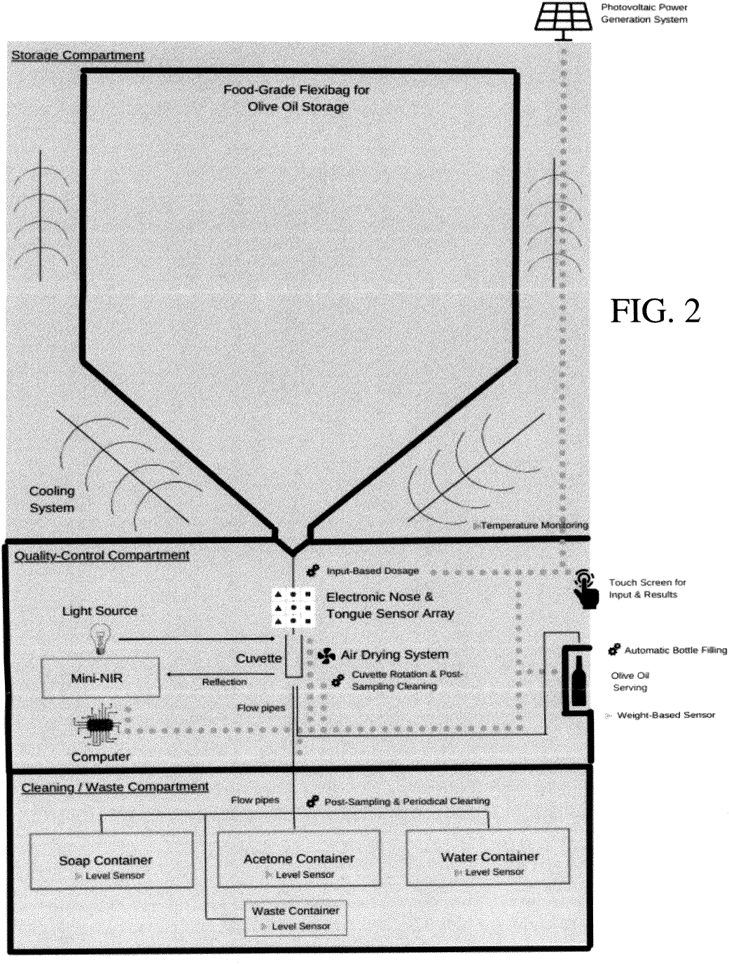

FIG. 2 is a schematic drawing of an exemplary real-time food product quality control system in accordance with an embodiment of the present disclosure. FIG. 2 illustrates an exemplary real-time food product quality control system as provided at least by the system 100 in accordance with the present disclosure. The system may comprise a plurality of components that contribute to providing a complete food product (e.g., a complete olive oil food product) quality assurance (QA) and/or quality control (QC) solution. Components of the system may be subdivided into the following functional categories: storage, quality control, network, and consumer experience.

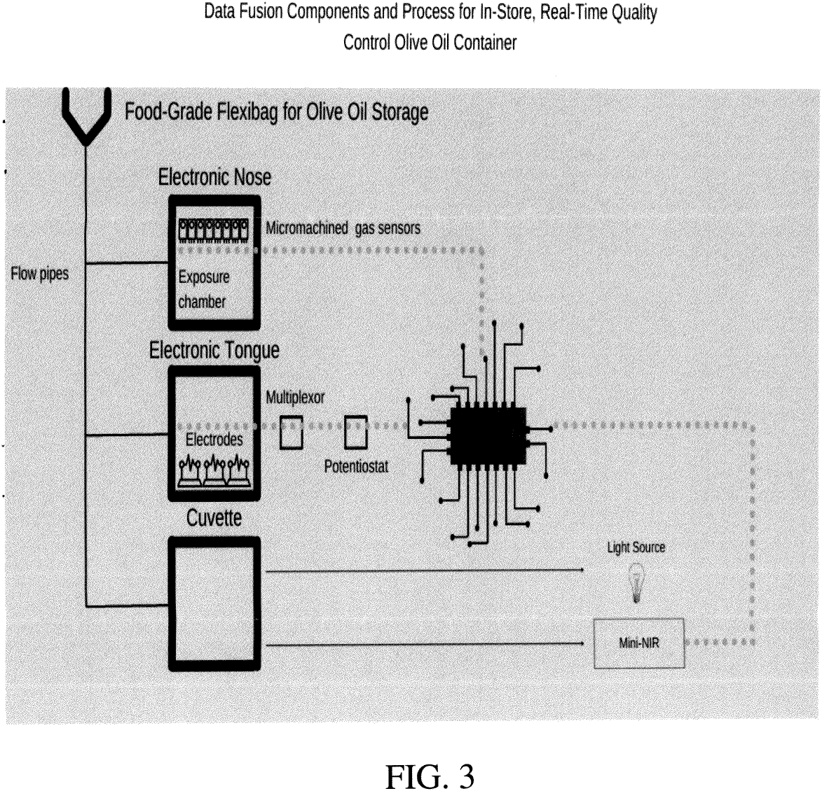

FIG. 3 is a schematic drawing of an exemplary quality control feature in accordance with an embodiment of the present disclosure. FIG. 3 illustrates an exemplary quality control feature as provided at least by the quality control device 108 in accordance with the present disclosure. The quality control feature may comprise a plurality of quality control methods (e.g., NIR spectroscopy, electronic nose, electronic tongue) the feedback of each may be analyzed by the quality control application 104 in accordance with an embodiment of the present disclosure.

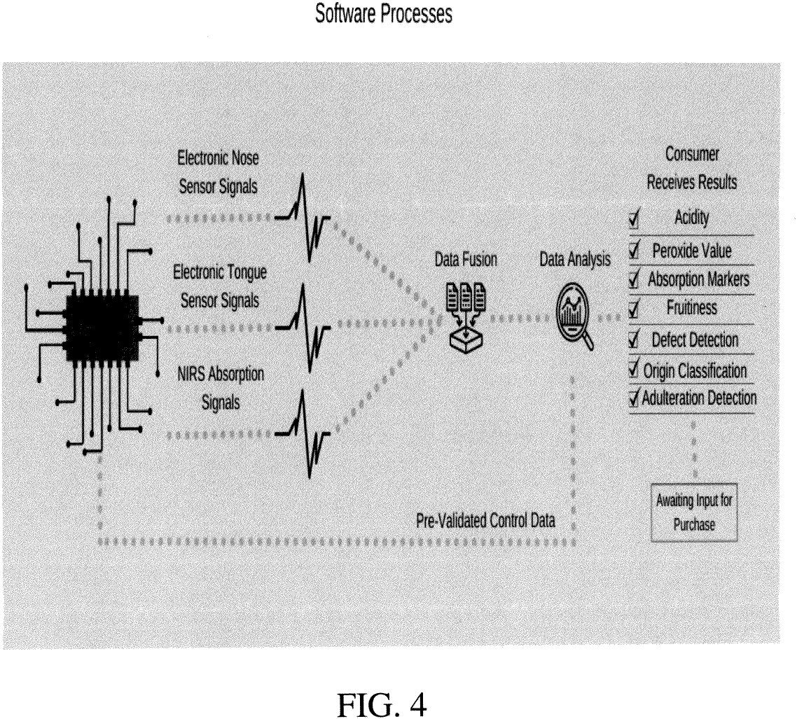

FIG. 4 is a schematic drawing of an exemplary data fusion and data analysis method in accordance with an embodiment of the present disclosure. FIG. 4 illustrates the process of result generation as provided at least by the quality control server 102 and the quality control application 104 in accordance with the present disclosure. The result generation process may comprise the receival of data from the quality control device 108, the integration of said data in homogenous data sets, the comparison of generated data sets with a known database 112 stored in the quality control server 102, and the final data analysis for the generation of results.

The foregoing descriptions of specific implementations have been presented for purposes of illustration and description. They are not intended to be exhaustive or to limit the invention to the precise forms disclosed, and modifications and variations are possible in view of the above teaching. The exemplary implementations were chosen and described to best explain the principles of the invention and its practical application, to thereby enable others skilled in the art to best utilize the invention and its implementations with modifications as suited to the use contemplated.

Systems and methods have been shown and described in the most practical and exemplary implementations. Departures may be made which fall within the scope of the invention. Optimal features of the invention include variations in size, materials, shape, form, function, manner of operation, assembly, and use. All structures, functions, and relationships equivalent or essentially equivalent to those disclosed are intended to be encompassed by the invention.

In an embodiment, a method for assuring quality control of food products is provided. The method comprises a computer directing a storage device to dispense a first quantity of a food product. The method also comprises the computer directing a quality control device to perform at least one test on the dispensed product. The method also comprises the computer directing a networking device to transmit instructions regarding at least maintaining and monitoring the storage device and quality control device. The method also comprises the computer directing a consumer experience device to provide details describing the at least one test.

The method also comprises the computer directing replacement of the storage device upon detecting an empty condition of the storage device, the storage device comprising a flexi-bag. The method also comprises the computer directing the quality control device to measure at least one characteristic of the dispensed food product.

The method also comprises the computer activating at least one of near-infrared spectroscopy, electronic nose, and electronic tongue apparatus of the quality control device. The method also comprises the computer configuring the quality control device to direct the apparatus to function without consumables, spare parts, or maintenance.

The method also comprises the computer comparing results of the at least one test with data in a database describing reference measurements of the at least one characteristic. The method also comprises the computer directing a sensor system to oversee sampling and cleaning of the storage device.

The method also comprises the computer providing a touch-screen device as the consumer experience device. The method also comprises the computer directing the touch-screen device to present information at least one of describing available quantities of the food product to order, describing details of recent analyses of the food product, describing origins of the food product, describing processing of the food products, describing persons associated with production of the product, and describing expertise of the persons in their field.

In another embodiment, a food quality control system is provided comprising a computer and an application executing thereon. The system receives results of a test of quality of a first food product, accesses a database describing quality standards and requirements for the food product, determines, based on analysis of the results with respect to the standards and requirements, that replacement of the food product is indicated, and transmits directions to a quality control device to replace the food product.

The food product is stored at a retail location. The test is performed by at least one of a near-infrared spectroscope, an electronic nose apparatus, and an electronic nose apparatus.

The food product is olive oil. The food product is stored in and dispensed to consumers from a flexi-bag.

Information describing storage, describing quality control, and describing origins of the food product are available for viewing by users of touch-screen devices located one of remotely from and proximate the retail location.

In yet another embodiment, a system for receiving information about and transacting for food products comprising a client device and application executing thereon. The system transmits an inquiry about availability and quality of a food product at a first retail location. The system also receives from a remote server a report describing quality of the food product, receives entry of an order, based at least on availability and the report, for a first quantity of the food product from the retail location, and displays confirmation of transaction details associated with the order.

The food product is olive oil. The report further describes origin of the food product.

The quality of the food product is verified at the retail location by at least one of a near-infrared spectroscopy apparatus, an electronic nose apparatus, and an electronic nose apparatus. The report further describes cleaning and storage of the food product.

What is claimed is:

1. A method for assuring quality control of food products, comprising:
a computer directing a storage device to dispense a first quantity of a food product;
the computer directing a quality control device to perform at least one test on the dispensed product;
the computer directing a networking device to transmit instructions regarding at least maintaining and monitoring the storage device and quality control device; and
the computer directing a consumer experience device to provide details describing the at least one test.

2. The method of claim 1, further comprising the computer directing replacement of the storage device upon detecting an empty condition of the storage device, the storage device comprising a flexi-bag.

3. The method of claim 1, further comprising the computer directing the quality control device to measure at least one characteristic of the dispensed food product.

4. The method of claim 1, further comprising the computer activating at least one of near-infrared spectroscopy, electronic nose, and electronic tongue apparatus of the quality control device.

5. The method of claim 4, further comprising the computer configuring the quality control device to direct the apparatus to function without consumables, spare parts, or maintenance.

6. The method of claim 1, further comprising the computer comparing results of the at least one test with data in a database describing reference measurements of the at least one characteristic.

7. The method of claim 1, further comprising the computer directing a sensor system to oversee sampling and cleaning of the storage device.

8. The method of claim 1, further comprising the computer providing a touch-screen device as the consumer experience device.

9. The method of claim 8, further comprising the computer directing the touch-screen device to present information at least one of describing available quantities of the food product to order, describing details of recent analyses of the food product, describing origins of the food product, describing processing of the food products, describing persons associated with production of the product, and describing expertise of the persons in their field.

10. A food quality control system, comprising:
a computer and an application executing thereon that:
receives results of a test of quality of a first food product,
accesses a database describing quality standards and requirements for the food product,
determines, based on analysis of the results with respect to the standards and requirements, that replacement of the food product is indicated, and
transmits directions to a quality control device to replace the food product.

11. The system of claim 10, wherein the food product is stored at a retail location.

12. The system of claim 10, wherein the test is performed by at least one of a near-infrared spectroscope, an electronic nose apparatus, and an electronic nose apparatus.

13. The system of claim 10, wherein the food product is olive oil.

14. The system of claim 10, wherein the food product is stored in and dispensed to consumers from a flexi-bag.

15. The system of claim 10, wherein information describing storage, describing quality control, and describing origins of the food product are available for viewing by users of touch-screen devices located one of remotely from and proximate the retail location.

16. A system for receiving information about and transacting for food products, comprising:
a client device and application executing thereon that:
transmits an inquiry about availability and quality of a food product at a first retail location,
receives from a remote server a report describing quality of the food product,
receives entry of an order, based at least on availability and the report, for a first quantity of the food product from the retail location, and
displays confirmation of transaction details associated with the order.

17. The system of claim 16, wherein the food product is olive oil.

18. The system of claim 16, wherein the report further describes origin of the food product.

19. The system of claim 16, wherein the quality of the food product is verified at the retail location by at least one of a near-infrared spectroscopy apparatus, an electronic nose apparatus, and an electronic nose apparatus.

20. The system of claim 16, wherein the report further describes cleaning and storage of the food product.
